(19) United States
(12) Patent Application Publication
(10) Pub. No.: US 2003/0040729 A1
(43) Pub. Date: Feb. 27, 2003

(54) Absorbent structure and method of producing the same

(76) Inventors: Krzysztof D. Malowaniec, Heidenheim (DE); Rainer Mangold, Herbrechtingen (DE); Thomas Wurster, Heidenheim (DE)

Correspondence Address: William M Hanlon Jr, Young & Basile, Suite 624, 3001 West Big Beaver Road, Troy, MI, 48084, US

(21) Appl. No.: 10/220,530

(22) PCT Filed: Mar. 2, 2001
(86) PCT No.: PCT/EP01/02385

(51) Int. Cl. A61F 13/15

(52) U.S. Cl. 604/368; 604/370

(57) ABSTRACT

The invention relates to an absorbent structure (30, 38, 50, 58, 62, 66) that is produced on the basis of superabsorbent polymer materials. Said superabsorbent polymer materials are interlinked by means of a thermoplastic polymer. The inventive structure is produced by extruding the superabsorbent polymer materials and the thermoplastic polymer while adding a blowing agent.

### DESCRIPTION

The invention relates to an absorbent structure formed on the basis of granular, superabsorbent polymer materials, where the superabsorbent polymer materials are bonded together by a lower-melting thermoplastic polymer. The invention relates additionally to a method for producing an absorbent structure of this kind and a hygiene article having such an absorbent structure as an absorbent element layer.

When an absorbent structure on the basis of superabsorbent polymer materials is mentioned in what precedes, this is understood to mean a structure having a content of more than 70% by weight of superabsorbent polymer materials.

It has been shown that absorbent element structures having such a high content of superabsorbent polymer materials (SAP) cannot be realized using traditional fiber-based structures, because the granular SAP materials cannot be arranged to be sufficiently accessible on the one hand and at the same time be immobilized on the other.

From DE-A-2 222 780 it is known to apply the granular superabsorbent polymer materials on an underlayer together with particles of a thermoplastic material to produce an absorbent structure formed on the basis of superabsorbent polymer materials and then to melt the thermoplastic material to obtain a composite.

A structure of this type of superabsorbent polymer materials and thermoplastic polymers was not successful in practice, because the accessibility of the superabsorbent materials for the impinging fluid was not sufficiently ensured. Moreover, this structure proved to be too rigid and was consequently characterized by insufficient comfort for the wearer.

With this as the point of departure, the object of the invention is to obviate the aforementioned disadvantages in the case of an absorbent structure of the generic type named at the beginning having a high SAP content, that is, to achieve a flexible structure having good fluid absorption and retention characteristics, which can in addition be manufactured simply.

This object is achieved under the invention in the case of a conventional absorbent structure by extruding the superabsorbent granular polymer materials and the thermoplastic polymer while adding a blowing agent.

The production of an open-cell polypropylene foam with a pore content of more than 20% by volume is known from WO 94/13460. Packaging and the use of the foam for sound absorption and thermal insulation are named as areas of application.

The production of an extruded thermoplastic foam is also known from WO 98/56430. The foam preferably has a structure consisting of cell walls and cells. The foam can be used, according to the description, as the container for receiving and retaining a piece of meat or as a layer in a diaper.

With the present invention the proposal is made for the first time to bond granular, particulate superabsorbent polymer material by means of at least partially molten thermoplastic polymer materials by extruding the mixture which is subject to pressure and temperature while adding a blowing agent. It is hereby possible on the one hand to immobilize, that is, fixate the granular superabsorbent polymer materials within the structure and on the other to form a structure which can be penetrated extremely well by an impinging fluid, such as urine for example. It was shown that the fluid can penetrate very quickly into the open-pore foamed structure formed through extrusion of the mixture as the blowing agent expands and can reach the superabsorbent polymer materials contained therein, where it is then permanently retained. It also turned out that, to a far lesser degree, the swellable, superabsorbent polymer materials in the structure under the invention cause the so-called gel blocking effect which proves to be problematic with higher weight percentages of swellable polymer materials in absorbent fiber structures, because the polymer materials swelling in the fluid compress the interstices between fibers so that no capillarity is left to carry the fluid into still unutilized absorbent element areas. An additional problem in the case of absorbent structures formed from natural fibers is their tendency to collapse in a saturated condition, said problem also being described as wet collapse. This also leads to a reduction of the ability to distribute fluid within an absorbent structure. In the case of the extruded absorbent structure under the invention, the problems discussed in what preceded do not occur, or occur to a far lesser degree, for which reason the absorption capability of the superabsorbent polymer materials is available almost in its entirety to absorb the impinging fluid even at very high concentrations of more than 70% by weight.

The granule size of the particles of superabsorbent polymer materials is in the normal range, and the mass median is preferably about 200-800 microns, where preferably no more than 20% by mass of the particles are smaller than 200 microns; in this respect reference is made to the disclosure in U.S. Pat. No. 5,061,259.

The extruded open-pored structure exhibits a retention capacity of at least 10 g of fluid per gram of the extruded structure. Absorption capacity can be determined in a test procedure to be described later in greater detail.

In a further embodiment of the invention, the percentage by mass of the thermoplastic polymer is less than 20% by weight, and specifically less than 10% by weight of the absorbent structure.

A polymer from the group of polyolefins, specifically polypropylenes and/or polyethylenes, has proved in a particularly preferred way to be the thermoplastic polymer, which quasi forms the binding agent for the superabsorbent particulate polymer materials. Corresponding copolymers, specifically ethylene vinyl acetate copolymers, as well as halogenated polyolefins can be used. In principle, however, other thermoplastic polymers are suitable for the manufacture of the inventive absorbent structure, for example, those from the group of styrene polymers.

In order to make available as great a fluid absorption volume as possible and to expose as great a surface of the superabsorbent polymer materials as possible for fluid absorption, the degree of foaming is at least 50%, preferably it is higher than 100%. The degree of foaming of the structure is defined as the volumetric increase of a mass unit of the mixture in a state inside the extrusion apparatus on the one hand, or in the extruded state of the finished structure on the other hand.

In an advantageous manner the absorbent structure can comprise between 3% and 20%, preferably between 5% and 10% by weight of fibers as additives. They can be natural or synthetic fibers, preferably polyester fibers, but whose melting or degradation temperature is higher than the melting temperature of the related thermoplastic polymer inside the extrusion apparatus. The effect of the fibers is that passages are formed during the extrusion process which promote the penetration of aqueous fluid into the structure.

In a particularly advantageous way the invention allows absorbent structures to be formed whose basic weight varies in the longitudinal direction and/or in the transverse direction of the structure, where the longitudinal direction corresponds to the direction of extrusion. By suitably shaping an extrusion opening, specifically an extrusion slit, any kind of cross-sectional structures can be achieved. Thus, viewed particularly in cross-section perpendicular to the longitudinal direction, the thickness of the absorbent structure could be greater in the center and, corresponding to the shape of the extrusion opening, could decrease in any fashion at all toward the sides.

Like all the absorbent structure to be explained in what follows, the structure can comprise in addition a surfactant substance, specifically a hydrophilizing agent in an amount of preferably 0.2%-10%. The already extruded structure can be secondarily contacted with the hydrophilizing agent. Preferably this agent is fed to the extruder together with the remaining initial materials or injected into the already molten polymer mass, so it is already present commingled with the polymer melt before it is extruded.

Advantageously alkyl sulfonates, fatty acid derivatives or fluorine chemicals are used for this—as described in the publication “Polymer Melt Additives: Their Chemistry, Structure and Uses,” (authors Gasper et al., lecture during Insight 1999—Nonwovens Business/Fiber & Fabric Conferences, San Diego, Calif., 1-2 Nov. 1999. Proceedings published by Marketing Technology Services, Inc.).

Protection is also sought with this invention for a disposable, absorbent hygiene article, specifically a diaper, sanitary napkin or an incontinence pad, having a specifically multi-layer absorbent element which is characterized by an absorbent element layer made of an absorbent structure of the previously described inventive type.

This absorbent element layer can be located on the side of a fluid distribution and intermediate retention layer facing outwardly from the body. It is also conceivable that the fluid distribution and intermediate retention layer which comprises fewer or no superabsorbent polymer materials, is also manufactured as an extruded, foamed structure. In this case, both absorbent elements could be produced inside the manufacturing machinery by extrusion and be placed one on top of the other to create the composite layer. Direct coextrusion of both layers, i.e. production by the same extrusion apparatus, is conceivable and advantageous.

It is furthermore additionally possible to configure the inventive absorbent SAP-containing structure itself in multiple layers. For example, a first layer facing outwardly from the body can be overlaid by a second body-facing layer. In such a case, the absorbent SAP-containing structure can, for example, be furnished with an advantageous SAP profile. In particular, the first layer facing outwardly from the body can contain less SAP (in percent by weight relative to the first layer) than the second body-facing layer. It can be advantageous that the surface extent, that is the width and/or length, of the first layer facing outwardly from the body is different from the surface extent of the second body-facing layer, specifically it can be advantageous to configure the first layer facing outwardly from the body larger, specifically wider with respect to its surface extent than the second body-facing layer. This multi-layer construction of the absorbent SAP-containing structure itself can be produced simply, by direct coextrusion of the layers.

Furthermore, it would be conceivable that a layer impervious to fluids facing outwardly from the body, which is normally formed of a pre-manufactured plastic film, is produced by coextrusion with the absorbent element. In this case, it would prove to be advantageous and expedient to configure all three aforementioned layers, or even additional layers by coextrusion by means of a single co-extrusion apparatus inside the manufacturing machinery. A fixative means, such as a hot melt adhesive for example, can then be advantageously omitted, since the extruded layers can be fixed in position with each other and also with respect to additional layers and/or elements in the course of their manufacture.

It is generally noted that the fluid distribution and intermediate retention layer, which contains very little or even no superabsorbent polymer materials, can be additionally configured and manufactured in the same way as the inventive absorbent structure or the aforementioned absorbent element. It can have additives in the form of fibers or surfactant substances and be configured manufactured with a varying thickness or varying basic weight respectively.

As already mentioned in what preceded, it can prove to be advantageous if the absorbent element has a varying thickness in the longitudinal direction of the article or in the transverse direction, that is, if it is configured with a profiled shape. Through an agglomeration of material in a central area of the hygiene article, the fluid absorption capacity available there can consequently be configured with any profile in and of itself, specifically bell-curve shaped or graduated.

In an especially advantageous embodiment of the invention it is also possible that the absorbent element has upwardly extending wall sections on both sides running in the longitudinal direction of the article and toward the wearer which form a leakage barrier. These wall sections assume the function of gatherings extending upwardly in the direction of the wearer which are normally formed in known hygiene articles from nonwoven materials with inserted means of elastification.

Of course it is understood that wall sections of this kind can also run in the transverse direction and can also exercise a blocking effect there, particularly for separating solid and liquid body excretions.

Subject of the present invention is also a method for producing an absorbent structure, specifically in accordance with claims 1 to 10 using the following process steps:

introduction of a thermoplastic polymer into an extrusion apparatus,

introduction of a superabsorbent particulate polymer material in granulate form into the extrusion apparatus, where the percentage content by weight of the superabsorbent polymer material to the thermoplastic polymer is at least 70% by weight,

introduction of a blowing agent under positive pressure,

extrusion of the mixture, where the blowing agent results in foaming of the thermoplastic polymer which bonds the granular polymer materials together to form a matrix when pressure is reduced.

CO₂ is preferably used as the blowing agent, although equally conceivable would be saturated, unsaturated, cyclic hydrocarbons and halogenated hydrocarbons as well as noble gases such as argon, helium or nitrogen or a water/air mixture.

Inside the extrusion apparatus positive pressure is preferably developed high enough that the blowing agent finds itself in a so-called supercritical state, in which the phase boundary between the fluid and gaseous aggregate state disappears and only a single homogenous phase is present. In the case of CO₂ this state is present at temperatures above about 31° C. and pressures above 73.5 bar. In this state the blowing agent can be mixed optimally for preparing a physical foaming process with the superabsorbent polymer materials and with the molten thermoplastic polymer. If this mixture is then passed through an extrusion die into an area of lower pressure, the blowing agent vaporizes with decreasing temperature, and the foamed open-pore structure results.

Since not only a preferably supercritical state of the blowing agent has to be attained, but the thermoplastic polymer also has to be at least partially melted, temperatures of 80° C. to 200° C. are generated inside the extrusion apparatus.

In an especially advantageous improvement to the invention, moist superabsorbent polymer material can be used to produce the structure in accordance with the invention, whose moisture content is at least 1% by weight, preferably at least 4% by weight. In this case, the fluid content can additionally act as a blowing agent.

In a quite particularly advantageous improvement to the invention, moist superabsorbent polymer material whose moisture content is at least 1% by weight, preferably at least 4% by weight, can be used for producing the inventive structure, said polymer material. In this case the fluid content can be used as an additional blowing agent.

The extrusion cross-section is changed during extrusion to produce varying thickness or shape in the longitudinal or transverse direction of the structure being produced. If a large number of similarly configured structures is to be extruded, it proves to be advantageous if the extrusion cross-section is changed in a correspondingly oscillating fashion. This takes place transversely to the direction of extrusion, specifically in the discharge direction, whereby the thickness of an extruded web is varied, or transversely to the discharge direction, whereby its width is varied.

In order to increase the accessibility of the extruded structure for aqueous fluids, it is advantageous to expose the extruded structure to additional mechanical treatment, for example, stretching, compression (rolling) and/or perforation by means of a fine needling tool.

Multi-stage rolling of the extruded structure is particularly advantageous. Multi-stage rolling enables the application of several temperature and/or pressure stages. In this way the extruded structure can be changed/optimized more selectively with respect to the requirements of its later use. Thus it has proved to be advantageous to compress the extruded structure in a first calendering stage at a temperature which is suitable for maintaining the thermoplastic polymer in the extruded structure above its softening point. Depending on the polymer employed, a temperature in the calendering stage of 40° C.-90° C., specifically 45° C.-75° C., specifically 50° C.-60° C., has been shown to be suitable. Afterwards the extruded absorbent structure can be advantageously compressed cold in a second calendering stage, which is performed specifically at temperatures of 0° C.-30° C., specifically at 15° C.-25° C.

It has furthermore proved to be advantageous to subject the extruded structure additionally to stretching.

It proves to quite particularly advantageous if the inventive method is integrated into a production process for hygiene articles and thereby an absorbent element is extruded directly inside a machine. In such an event, fiber forming and discharge stations can be dispensed with in the manufacturing machinery (at least for the extruded absorbent element). As already mentioned, several absorbent elements which are to be positioned one above the other can be produced in the same machinery.

Additional details, features and advantages of the invention can be found in the appended patent claims and from the drawing and the description which follows of a manufacturing apparatus, of the manufacturing process as well as of several embodiments of inventive absorbent structures. In the drawings:

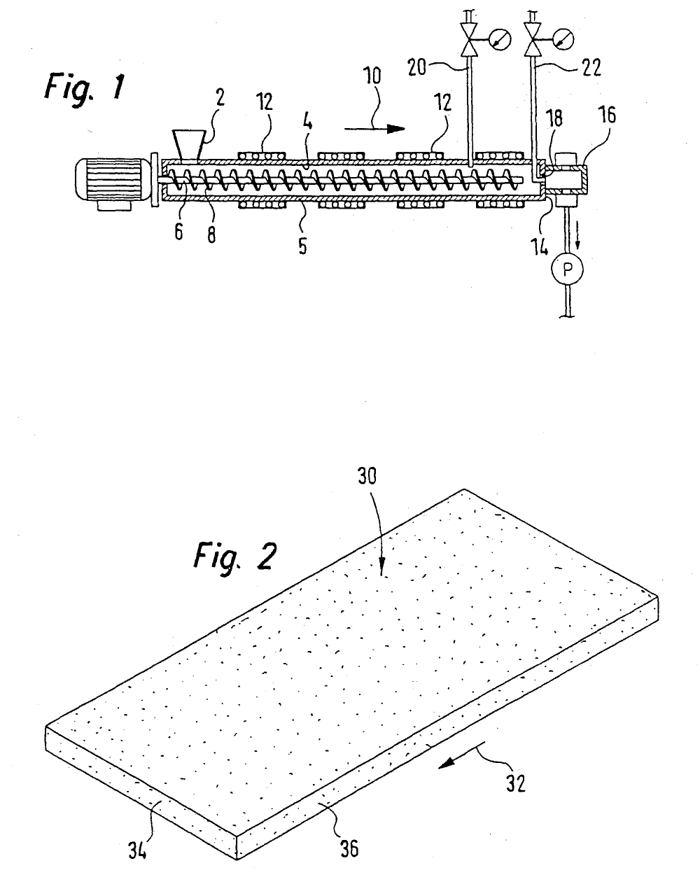

FIG. 1 shows a schematic view of an apparatus for producing an inventive absorbent structure;

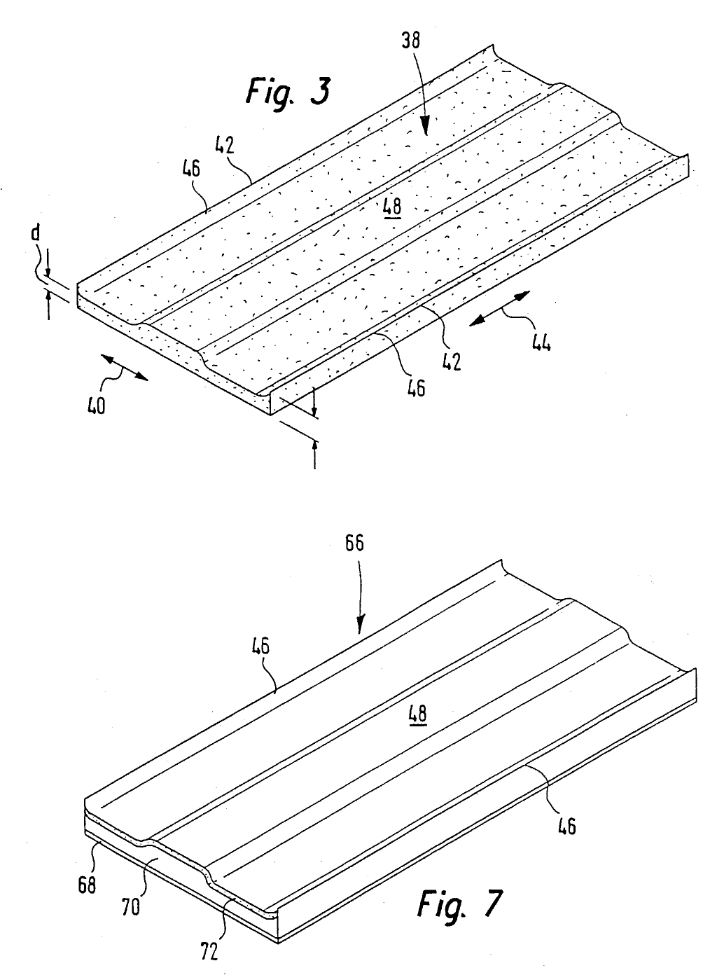

FIGS. 2 to 6 show different embodiments of inventive absorbent structures;

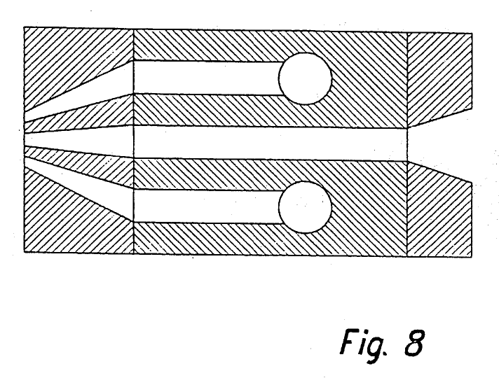

FIG. 7 shows an additional embodiment of a multi-layer inventive absorbent structure and FIG. 8 shows a schematic representation of a co-extrusion apparatus.

FIG. 1 shows an apparatus for producing an inventive absorbent structure. The apparatus comprises a funnel-shaped feed mechanism 2 through which a solid-matter mixture, which was preferably produced in advance in accordance with the by-weight percentile composition of the individual components, can be fed into a cylindrical interior 4 of a high-pressure stable tubular housing 5 of the production apparatus. A shaft 6 extends in this interior 4 having a helical screw 8 driven by an electric motor 6. When the shaft 6 is driven, the solid matter mixture which was introduced is further mixed and transported in longitudinal direction 10. Heating devices 12 are provided on the outer circumference of the tubular housing 5.

An extrusion tool 16 can be mounted on the end face 14 of the tubular housing 5 at the end opposite the feed device 2. The extrusion tool 16 communicates through an opening 18 on the end face 14 with the interior 4 of the tubular housing.

Injection devices 20, 22 discharge into the interior 4, whereby they discharge quasi inside the opening 18. A blowing agent under operating pressure can be introduced into the interior 4 through the injection devices 20, 22. In this way an operating pressure can be set and maintained in the interior 4 during the extrusion process, generally above 70 bar depending on the blowing agent employed in the extrusion process.

To produce an inventive absorbent structure a polyolefin, specifically a polypropylene and/or polyethylene granulate, for example, can be used as a thermoplastic polymer. This granulate is mixed with swellable superabsorbent polymer materials, which are adequately known in combination with absorbent layers in hygiene articles and therefore do not need to be described in greater detail. The mixture obtained in this way is transported into the interior 4 by means of the conveying device 2. The mixture is brought up to an operating temperature by the heating devices 12 such that the thermoplastic polymer melts, but the particulate superabsorbent polymer materials are not affected in the slightest.

A blowing agent, for example CO₂, is introduced into the interior 4 through the said injection devices 20, 22 so that an operating pressure obtains there which is suitable for extruding the partially molten mixture via the extrusion tool 16. Since the blowing agent is intended to result in foaming of the thermoplastic polymer, it is preferably introduced into the interior 4 in the so-called “supercritical stage.”

When the mixture obtained in this way passes through the extrusion die of the extrusion tool 16, the blowing agent expands as result of the accompanying drop in pressure and the mixture is foamed, that is to say, pores or cavities which communicate with each other are formed by the expanding and usually escaping blowing agent. The granulate superabsorbent polymer materials are bound in place inside this cavity structure formed by the hardening of the thermoplastic polymer. They are immobilized, but their surface is thereby exposed through the cavities created as a result of the extrusion process and the expansion and escape of the blowing agent and is available to absorb fluid.

FIG. 2 shows a section of an extruded absorbent structure 30 which comprises 80% by weight superabsorbent polymer material and 13% by weight thermoplastic polymer, i.e. polyethylene (PE), and additionally 7% by weight polyester fibers (PES).

The direction of extrusion is identified by the arrow 32 so that the formed end surface with the reference numeral 34 represents the plane perpendicular to the direction of extrusion 32. The absorbent structure 30 is shown exactly rectangular in FIG. 2, it must be pointed out that only a basically plane surface can be obtained by an extrusion process, and even with a precisely rectangular extrusion die, rounded edges can be formed. However it would be possible to configure a continuous web in the direction of extrusion 32 with end surfaces 34 and longitudinal surfaces 36 exactly perpendicular to each other by lengthwise and crosswise trimming.

FIG. 3 shows an absorbent structure 38 which has a varying thickness d in the transverse direction 40. Running along both of its long edges 42 in the longitudinal direction 44, the structure has a wall area 46 extending upwardly, that is in the thickness direction, which terminates in a peak in the upward direction. From outside to inside, in the transverse direction 40, this wall area 46 falls off asymptotically and transitions into a plane section with constant thickness d and then rises again toward the center in accordance with the profile seen in FIG. 3 to a section 48 of greater thickness. A cross-sectional structure of this kind can be produced by shaping the extrusion die correspondingly.

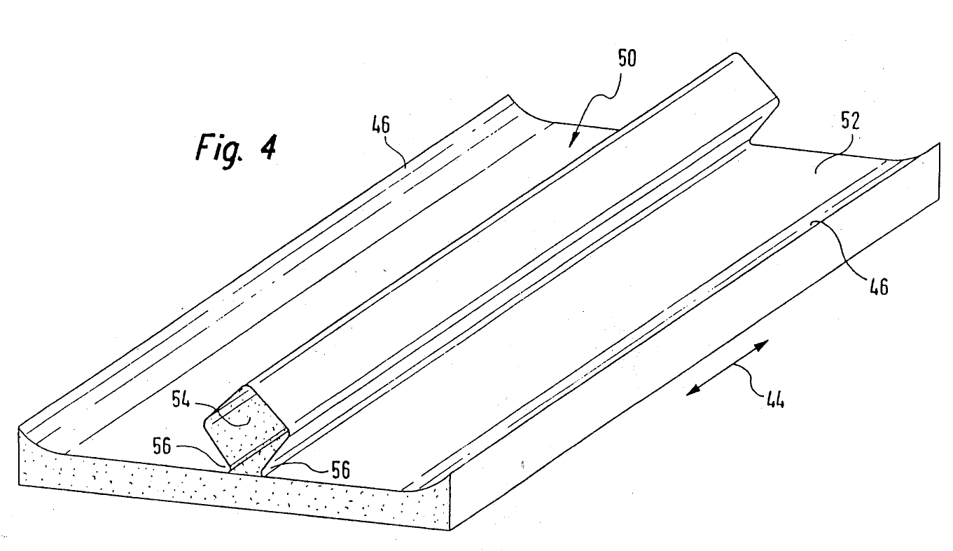

FIG. 4 shows a further embodiment of an inventive absorbent structure 50 having upwardly extending wall areas 46 on both sides running in the longitudinal direction 44 as in FIG. 3. The structure 50 has an area in the center also running in the longitudinal direction 44, essentially lozenge-shaped in cross-section and rising above a surface 52. Because of its lozenge-shaped cross-section, the area 54 forms undercuts 56 when viewed in the direction perpendicular to the surface 52. The creation of structures which are round, elliptical or polygonal in cross section, with or without undercuts, would be conceivable. Such absorbent element structures are intended for use in feminine hygiene products. The raised area 54, whatever geometric form it may have, can extend at least partially into the vagina when it is worn and thus create a direct contact between the vagina and the absorbent hygiene product.

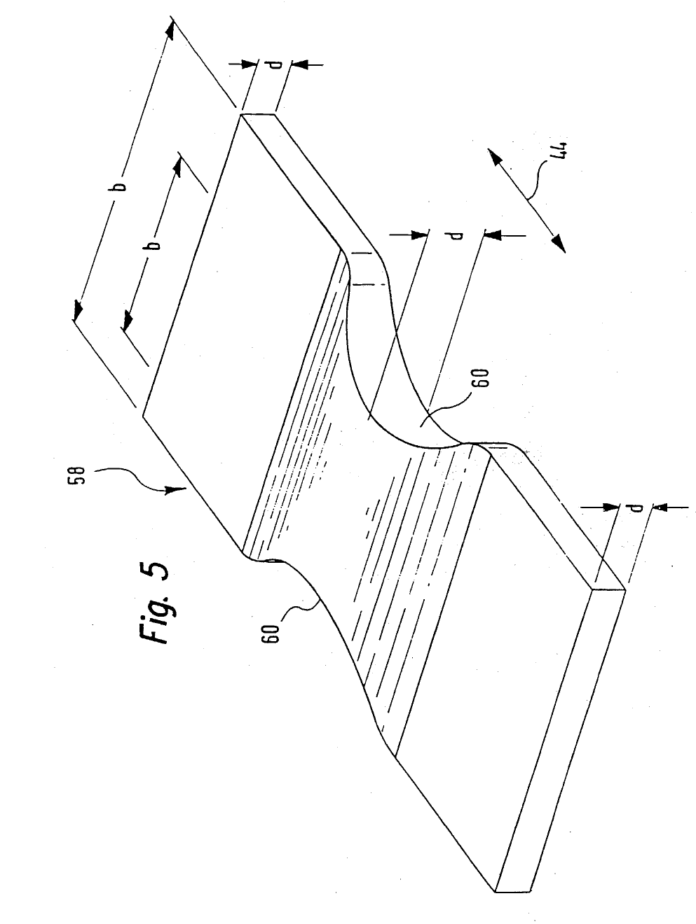

FIG. 5 shows in an appropriate view an absorbent structure 58 produced by extrusion having varying thickness d in the longitudinal direction of extrusion 44. Furthermore, the absorbent structure shown 58 has a varying width b in the longitudinal direction 44. The absorbent structure shown 58 would lend itself to the production of a diaper, whereby arcuate leg openings 60 are provided in the middle, and in this area forming the crotch of the diaper an agglomeration of material is given by the greater thickness d provided there.

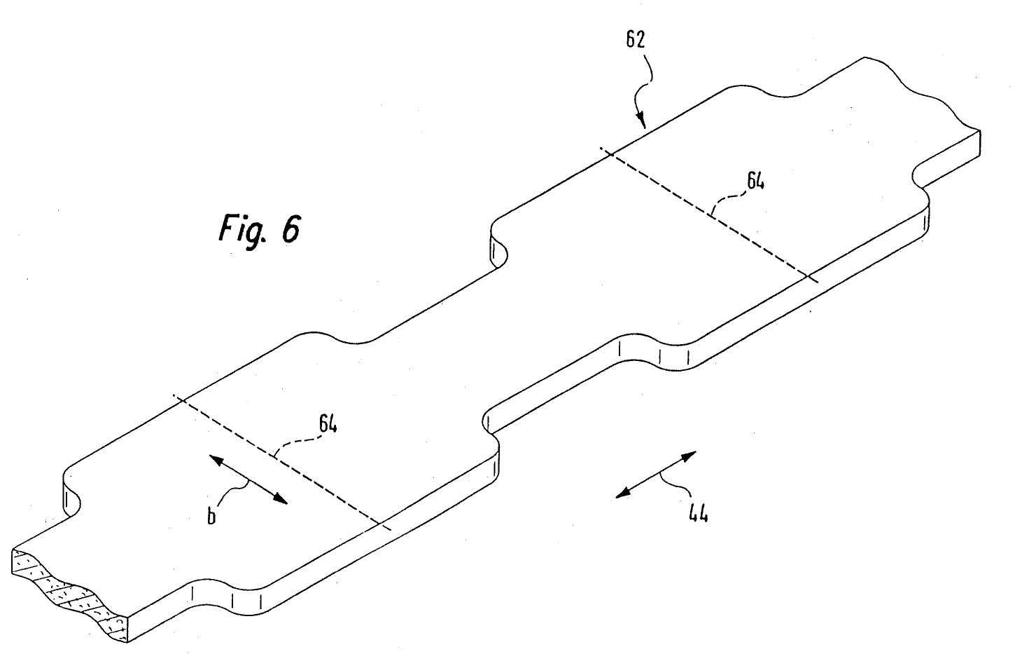

FIG. 6 shows schematically a merely suggested continuous extruded web 62 with varying width b in the longitudinal and extrusion direction 44. The broken lines 64 suggest the division of the continuous web by transverse cutting to create individual sections for the production of diapers.

FIG. 7 shows a continuous extruded absorbent structure 66, which is produced by the co-extrusion of three layers which is suitable for use in a hygiene article, in particular a diaper. The structure comprises a first lower extruded film layer 68 of PE and/or PP. A middle extruded layer 70 formed on the basis of superabsorbent polymer materials, which from its composition can correspond to the layer described in connection with FIG. 7, is identified with the reference numeral 70. A surface layer 72 on a polyester fiber (PES) base, free firstly of superabsorbent polymer materials and secondly of polyethylene and/or polypropylene (PE/PP), is furnished on its upper side. All three layers 68, 70, 72 are produced in a co-extrusion apparatus as shown schematically in FIG. 8, whereby to produce layers 70 and 72 a blowing agent under positive pressure was used to create an open-pore foamed structure through expansion and evaporation of the blowing agent. The structure 66 is configured in cross-section in accordance with FIG. 3; it has lateral upwardly extending wall areas 46 running in the longitudinal direction 44, which can act as a leakage barrier in a hygiene article and perform the function of ribbing normally formed on the basis of nonwoven materials. The agglomeration of material from a greater thickness of the absorbent layer 70 in a center area 48 makes available a greater fluid absorption capacity from greater quantities of superabsorbent polymer materials. The upper layer 72 facing the body functions as a fluid distribution and intermediate retention layer. This means it captures a great volume of fluid through its greater volume of pores when suddenly impacted by fluid, then distributes this fluid with a time-delay in the direction of its thickness, but also in a horizontal direction, and releases it to the retention layer 70 located below it.

The fluid retention capability of an inventive extruded absorbent structure with a content of least 70% by weight of superabsorbent polymer materials is determined by the centrifuge test to be described in what follows by giving its retention value. The absorbent structure to be tested is weighed in its dry state to determine its mass in grams. A plurality of specimens is immersed completely for 30 minutes in a 1-percent aqueous solution of sodium chloride of demineralized water as the test solution and then centrifuged for 4 minutes at 276 times the force of gravity. Then the specimens are weighed again to determine their mass including the fluid bound in them. The mass of the absorbed or bound fluid is therefore the difference between the mass determined after centrifuging and the dry mass of each of the specimens. If this difference m_{fl} is divided by the dry mass m_{dry}, the result is the retention value g_{fl}/g_{dry} in the unit.

What is claimed is:

1. Absorbent structure (30, 38, 50, 58, 62, 66) formed on the basis of superabsorbent polymer materials, where the superabsorbent polymer materials are bonded by a thermoplastic polymer, characterized in that the superabsorbent polymer materials are extruded while a blowing agents is added.

2. Absorbent structure (30, 38, 50, 58, 62, 66), wherein the structure has a retention capacity of at least 10 g/g.

3. Absorbent structure (30, 38, 50, 58, 62, 66) from claim 1 or 2, wherein the percentage by weight content of the thermoplastic polymer is less than 20% by weight of the absorbent structure.

4. Absorbent structure (30, 38, 50, 58, 62, 66) from claim 3, wherein the percentage by weight content of the thermoplastic polymer is less than 10% by weight of the absorbent structure.

5. Absorbent structure (30, 38, 50, 58, 62, 66) from one of the preceding claims, wherein the thermoplastic polymer comprises a polyolefin, specifically polypropylene and/or polyethylene.

6. Absorbent structure (30, 38, 50, 58, 62, 66) from one of the preceding claims, wherein the degree of foaming is more than 50%.

7. Absorbent structure (30, 38, 50, 58, 62, 66) in accordance with claim 6, wherein the degree of foaming is greater than 100%.

8. Absorbent structure (30, 38, 50, 58, 62, 66) from one or the preceding claims, wherein the structure comprises 3-20% by weight, specifically 5-10% by weight, fibers as additives.

9. Absorbent structure (30, 38, 50, 58, 62, 66) from one of the preceding claims, wherein its basic weight varies in the longitudinal and/or transverse direction.

10. Absorbent structure (30, 38, 50, 58, 62, 66) from one of the preceding claims, wherein a surfactant substance is introduced as an additive.

11. Absorbent hygiene article for one-time use, specifically diaper, feminine sanitary napkin, incontinence pad, having a specifically multi-layer absorbent body, characterized by an absorbent element layer of an absorbent structure (30, 38, 50, 58, 62, 66) from one or more of the preceding claims.

12. Hygiene article from claim 11, wherein the absorbent element layer (70) is arranged on the side of a fluid distribution and interim retention layer (72) facing away from the body.

13. Hygiene article from claim 12, wherein the fluid distribution and interim retention layer (72) comprises a thermoplastic polymer and is extruded while a blowing agent is added.

14. Hygiene article from claim 13, wherein the fluid distribution and interim retention layer (72) does not comprise any superabsorbent polymer materials.

15. Hygiene article from claim 13 or 14, wherein the fluid distribution and interim retention layer (72) demonstrates a degree of foaming greater than 50%, preferably greater than 100%.

16. Hygiene article from claims 13, 14 or 15, wherein the fluid distribution and intermediate retention layer (72) contains 1-20% by weight, specifically 5-15% by weight fibers as an additive.

17. Hygiene article from one of the claims 11-16 having a fluid-impermeable plastic film layer (68) furnished on the side of the absorbent element layer (70), wherein the film layer is extruded together with the absorbent element layer (70).

18. Hygiene article from one of the claims 11-17, wherein the absorbent element has a varying thickness in the longitudinal direction (44) of the article.

19. Hygiene article one of claims 11-18, wherein the absorbent element layer (68) varies in thickness in the transverse direction (40) of the article.

20. Hygiene article claim 19, wherein the absorbent element layer has wall sections (46) which form a leakage barrier on both sides, running in the longitudinal direction (44) of the article and projecting up toward the wearer.

21. Hygiene article from one of claims 11-20, wherein the absorbent element has a wall section running basically in the transverse direction of the article and projecting up toward the wearer.

22. Method for producing an absorbent structure from one or more of claims 1-10, comprising the following steps:
introduction of a thermoplastic polymer into an extrusion apparatus,
introduction of a superabsorbent granular polymer material into the extrusion apparatus, where the percentage by weight content of the superabsorbent polymer material to the thermoplastic polymer amounts to at least 70% by weight,
melting the thermoplastic polymer material at temperatures below a melting or degradation temperature of the superabsorbent polymer material,
introduction of a blowing agent under positive pressure,
extrusion of the mixture, whereby the blowing agent results in foaming of the thermoplastic polymer which bonds the granular polymer materials to form a matrix when pressure is reduced.

23. Method from claim 22, wherein CO₂ is used as the blowing agent.

24. Method in accordance with claim 22 or 23, wherein the thermoplastic polymer becomes molten at temperatures of 80 to 200 degrees Celsius.

25. Method in accordance with claims 22, 23 or 24, wherein superabsorbent polymers with a moisture content of at least 1% by weight, specifically of at least 4% by weight are used.

26. Method from one of the claims 22-25, wherein fibers are introduced into the extrusion apparatus as an additive.

27. Method from one of the claims 22-26, wherein a surfactant substance is introduced into the extrusion apparatus as an additive.

28. Method from one of the claims 22-27, wherein an extrusion cross section is changed during extrusion.

29. Method from claim 28, wherein the extrusion cross section is changed in an oscillating fashion.

30. Method from one of the claims 22-29, wherein the method is integrated into a production process for hygiene articles and therein the absorbent structure is extruded directly inside machinery for the high-speed production of hygiene articles.

31. Method from claim 30, wherein a double-layer absorbent element is formed inside the high-speed production machinery by co-extrusion of the layers, wherein the absorbent element comprises the absorbent structure (70) as an absorbent element layer and a fluid distribution and intermediate retention layer (72) on the body-facing side of said element layer.

32. Method from claim 31, wherein a triple-layer absorbent element is formed inside the high-speed production machinery by co-extrusion of the layers, wherein the third layer is a fluid-impermeable film (68) which is located on the side of the absorbent element layer facing away from the body.
